(19) United States
(12) Patent Application Publication
(10) Pub. No.: US 2002/0147177 A1
(43) Pub. Date: Oct. 10, 2002

(54) Formulation of artemisinin

(76) Inventors: Kah Hay Yuen, Penang (MY); Kit Lam Chan, Penang (MY); Ee Kiang Gan, Penang (MY); Jia Woei Wong, Penang (MY); Toh Weng Tuck, Penang (MY); David Sue San Ho, Ipoh (MY)

Correspondence Address: DAVID HO SUE SAN, 121, JALAN KUALA KANGSAR, IPOH, PERAK, 30010, MY

(21) Appl. No.: 09/768,134

(22) Filed: Jan. 24, 2001

(51) Int. Cl. A61K 31/724; C08B 37/16

(52) U.S. Cl. 514/58; 536/103

(57) ABSTRACT

A new formulation and process of producing a new formulation of artemisinin in the form of a complexation of artemisinin with beta-cyclodextrins. This new formulation of artemisinin having greater aqueous solubility, a higher dissolution rate and an improved bioavailability.

### TECHNICAL FIELD

This invention relates to a formulation of artemisinin with a better and more consistent absorption. More specifically, the invention is concerned with the formulation of a new dosage form of artemisinin, which is more absorbable and has an increased bioavailabiliry and its use in malarial patients at a lower dose level in comparison with commercial preparations.

### BACKGROUND ART

Artemisinin is the antimalarial principle isolated by Chinese scientists in 1972 from Artemisia annua L. It is a sesquiterpene with a peroxide bridge linkage with the peroxide moiety appearing, to be responsible for the antimalarial activity (Olliaro et al., 1995). Artemisinin is a fast acting blood schizonticide and is presently recommended for acute treatment of multidrug resistant malaria from Plasmodium falciparum as well as cerebral malaria (World Health Organization, 1994).

Artemisinin has poor aqueous solubility, and thus resulting in incomplete absorption after oral administration. This is due to a large fraction of the dose remaining undissolved for absorption upon reaching, the non-absorbable site in the large intestine. Under such conditions, the bioavailability can be increased by using,, a more water soluble formulation. Various more water or oil soluble derivatives from the parent compound have been synthesized but they either possess higher acute toxicity (artemether and arteether) or are unstable both within and outside the body (sodium artesunate and artesunic acid)l (Panisko et al., 1990; Li et al., 1998). Attempts to develop more water soluble, stable and bioavailable derivatives or formulation are still continuing.

Recently, we have discovered that a new formulation of arteminisinin with beta-cyclodextrins increases the bioavailability of artemisinin by approximately 180% with respect to that of a commercial preparation from Vietnam. The formulation consists of molecular complexation of artemisinin with beta-cyclodextrins, in which the beta-cyclodextrins will act as a host to accommodate the artemisinin molecule inside its cavity. The arteminisinin-beta-cyclodextrin complexes exhibit higher solubility and rapid dissolution and thus allowing the formulation to be more completely absorbed before reaching the non-absorbable site of large intestine. Moreover, the formulation may also circumvent the problem of recrudescence associated, with poor aqueous solubility, erratic absorption, short half-life and high first-pass metabolism.

### SUMMARY OF INVENTION

Accordingly the invention is said to broadly consist of a new formulation of artemisinin, in the form of a complexation of artemisinin with beta-cyclodextrins.

Preferably said formulation of artemisinin-beta-cyclodextrin complexes in the present invention has a greater aqueous solubility, higher dissolution rate and improved bioavailability when compared with the commercial preparation.

Preferably the more bioavailable formulations of artemisinin-beta-cyclodextrin complexes is used in the preparation of pharmaceuticals.

Preferably the use of the more bioavailable formulation as an antimalarial drug, would enchance its therapeutic efficacy, and thus requires a lower dose to be used which may reduce the incidence of recrudescence.

In another embodiment the invention may be said broadly to consist of a process for producing artemisinin in the form of a complexation of artemisinin with beta-cyclodextrins comprising of the following steps:

a) Mixing beta-cyclodextrins with distilled water.

b) Stirring the slurry of beta-cyclodextrins formed in step (a).

c) Adding finely ground artemisinin into the slurry.

d) Stirring the mixture formed in step (c) and then drying it at room temperature.

e) Grinding the dried product into fine powder and subsequently sieving it.

Preferably the slurry in step (a) consists of a ratio of 4 parts of beta-cyclodextrins to 5 parts of distilled water.

Preferably said slurry is stirred for 15 minutes. rate and improved bioavailability when compared with the commercial preparation.

Preferably the more bioavailable formulations of artemisinin-beta-cyclodextrin complexes is used in the preparation of pharmaceuticals.

Preferably the use of the more bioavailable formulation as an antimalarial drug, would enchance its therapeutic efficacy, and thus requires a lower dose to be used which may reduce the incidence of recrudescence.

In another embodiment the invention may be said broadly to consist of a process for producing artemisinin in the form of a complexation of artemisinin with beta-cyclodextrins comprising of the following steps:

a) Mixing beta-cyclodextrins with distilled water.

b) Stirring the slurry of beta-cyclodextrins formed in step (a).

c) Adding finely ground artemisinin into the slurry.

d) Stirring the mixture formed in step (c) and then drying it at room temperature.

e) Grinding the dried product into fine powder and subsequently sieving it.

Preferably the slurry in step (a) consists of a ratio of 4 parts of beta-cyclodextrins to 5 parts of distilled water.

Preferably said slurry is stirred for 15 minutes.

Preferably in step (c) 1 part of artemisinin (sieved through 300 μm mesh) is added into the said slurry.

Preferably in step (d) the mixture was stirred for 24 hours and dried by way of an extraction fan.

Preferably in step (e) the dried product being sieved through 300 μm mesh and the fine powder should have a loss on drying (LOD) of not more than 11.5%.

### BRIEF DESCRIPTION OF THE DRAWINGS

In the accompanying drawings:

FIG. 1 is a plot of the concentration of artemisinin and beta-cyclodextrin.

FIG. 2 is a plot of the time course of in vitro dissolution profiles of artemisinin-beta-cyclodextrin complexes physical mixtures and Artemisinin 250.

FIG. 3 is a plot of mean plasma artemisinin concentation and time profiles obtained with the complexes and Artemisinin 250.

FIG. 4a is a table containing the plasma artemisinin concentration of volunteers after dosing with Artemisinin 250 capsule.

FIG. 4b is a table containing the plasma artemisinin concentration of volunteers after dosing with artemisinin-beta-cyclodextrin complexes.

FIG. 5 is a table of numerical values of AUC_{0-∞}.

FIG. 6 is a table of numerical values of Cₘₐₓ.

FIG. 7 is a table of numerical values of Tₘₐₓ.

FIG. 8 is a table of logarithmic transformed AUC_{0-∞}values.

FIG. 9 is a table of logarithmic transformed Cₘₐₓ values.

FIG. 10 is a table of individual numerical values of kₑ.

FIG. 11 is a plot of mean absorption profiles of artemisinin from artemisinin in the complexes and Artemisinin 250.

FIG. 12 is a micrograph of the complexes and physical mixtures.

FIG. 13a & b are thermograms of the complexes and physical mixtures.

FIG. 14 is a molecular model of beta cyclodextrins with artemisinin complexes.

FIG. 15 is a percentage of parasites that remained in the blood versus time profiles.

FIG. 16 is a histogram displaying plasma levels at 1.5 and 3.0 hour after drug administration.

FIG. 17 is a plot of the mean fever subsidence versus time for the preparations.

### MODES OF CARRYING OUT THE INVENTION

The preferable method of carrying out this invention are discussed as follows:

Artemisinin was obtained commercially from China as the orthorhombic crystals form. In accordance with this invention, 16 g, of beta-cyclodextrins was mixed with 20 ml of distilled water. Slurry of beta-cyclodextrins is formed and stirred for 15 mins. 4 g of artemisinin is ground into fine powders (sieved through 300 μm mesh) before being) added into the slurry. The mixture is stirred for 24 hours and then dried under an extraction fan at room temperature. The dried product is then ground into fine powder and sieved through 300 μm (Endecotts Ltd., England). The fine powder should have a loss on drying (LOD) of not more than 11.5% (Mettler-LP 16, Mettler Toledo AG, Switzerland).

The artemisinin-beta-cyclodextrin complexes obtained were characterized and compared to either a physical mixture (to confirm that complexes is formed) or commercial preparation using methods such as solubility, dissolution, differential scanning, calorimetry (DSC), microscopic examination and in-vivo bioavailability. A physical mixture mentioned above is referred to a mixture of beta-cyclodextrins and artemisinin sieved through 300 μm separately.

The solubility of the artemisinin-beta-cyclodextrin complexes was evaluated in comparison with the crystals alone sieved through 300 μt mesh. 200 μg of each of the complexes and artemisinin crystals were separately shaken in 25 ml of water maintained at room temperature (25° C.) for five days. The solution was then filtered through a 0.2 μu membrane filter and suitably diluted before analysis. An analysis of the drug concentration in the solution was determined by high performance liquid chromatography using a method reported by Chan et al (1997). The complexes have a solubility of 173.22±2.75 μg /ml while the solubility of artemisinin crystals is 55.68±1.75 μg/ml, indicating that the solubility of the complexes is at least 3-fold higher than the crystals alone.

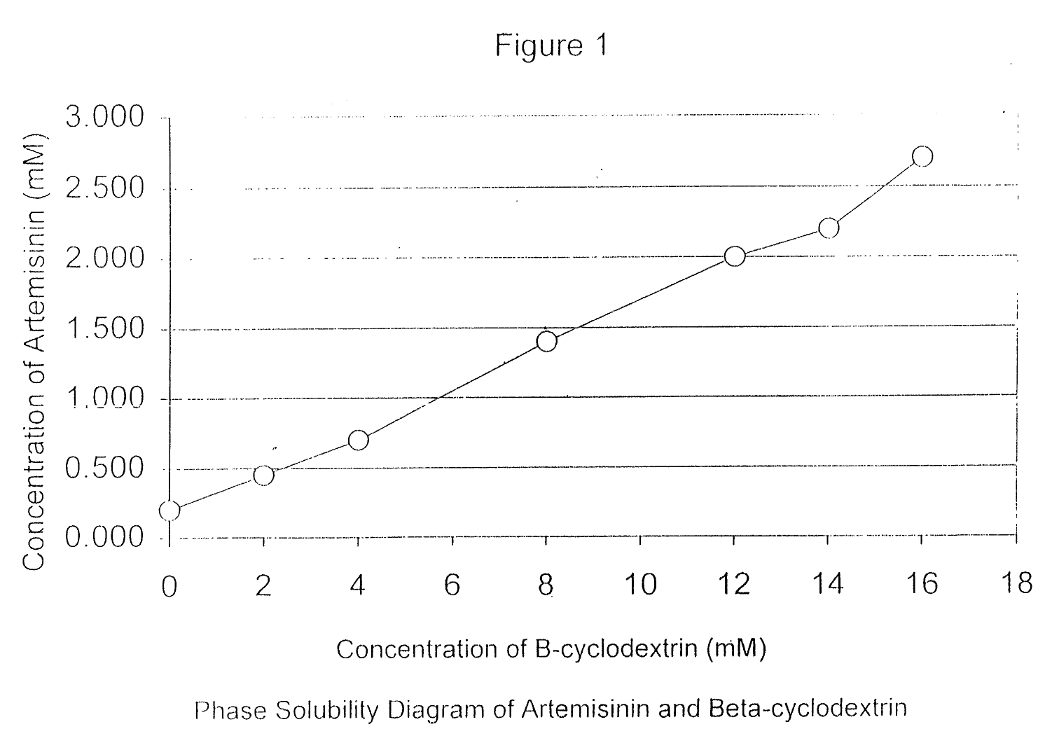

Phase solubility diagrams on artemisinin with beta-cyclodextrins were also constructed to determine the molar ratio of the complexes according to method by Higuchi et al (1965). Excess artemisinin was added into an increased concentration of beta-cyclodextrins and was shaken for 5 days at room temperature (25° C.). A straight line was obtained when the concentration of artemisinin dissolved was plotted against the concentration of beta-cyclodextrins, indicating that complexes were formed at a molar ratio of 1:1 as shown in FIG. 1. Stability constant was calculated and estimated to be 883.67 M⁻¹ at 25° C.

The in vitro dissolution of artemisinin-beta-cyclodextrins complexes, physical mixtures (as mentioned above) and commercial preparation (Artemisinin 250, Mekophar, Ho Chi, Minh, Vietnam) was determined under non-sink conditions, using the paddle method of the USP 23 dissolution test apparatus (Model PTWS3C, Pharma Test, Hainburg, Germany). The test was conducted with 150 mg of artemisinin for the complexes and physical mixtures while the commercial preparations employed a dose of 250 mg peer vessel. The dissolution medium was 900 ml of water maintained at 30° C. (room temperature) with the paddle rotation speed set at 100 rpm. Samples of 5 ml were collected at various intervals using an automated fraction collector, (SDX fraction collector, Sadex, Malaysia) over a period of 8 hours. The drug concentrations were measured by HPLC using an electrochemical detector at reductive mode after appropriate dilutions. Each test was repeated three times and the average concentration of artemisinin in solution versus time was calculated and plotted.

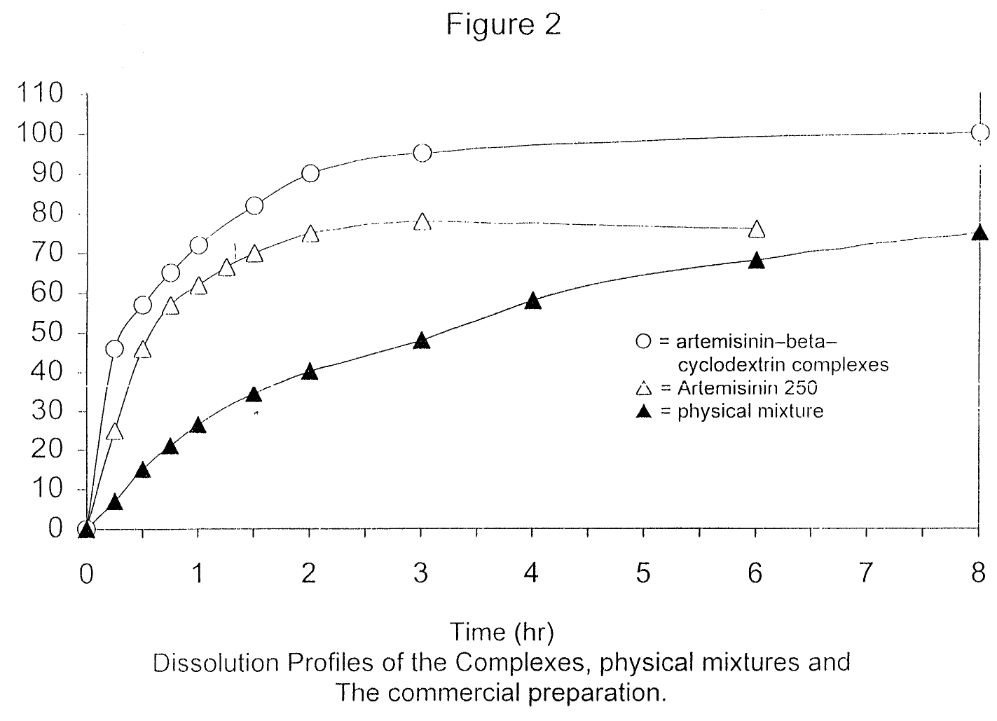

FIG. 2 shows in vitro dissolution profiles of artemisinin-beta-cyclodextrin complexes, physical mixtures and commercial preparation. The complexes showed a more rapid rate of dissolution, reaching saturation within 3 hours, whereas the physical mixtures do not show saturation even after 8 hours. Moreover, the saturation concentration of the complexes was markedly higher than the concentration of the physical mixtures at 8 hours, being approximately 100 μg/ml and 75 μg/ml, respectively. The commercial preparation (at a dose of 250 mg) indicated a slower dissolution compared to the complexes with a lower saturation concentration of 75 μg/ml, although a higher dose was used. Thus, the artemisinin-beta-cyclodextrins complexes has a higher solubility and faster dissolution than the commercial preparation.

A comparative in vivo bioavaiability study was conducted to investigate the bioavailability of the complexes at the dosage level of 250 mg with the commercial product, Artemisinin 250 mg (Mekophar, Ho Chi Minh, Vietnam). Twelve healthy adult male volunteers between 20 and 44 years old (mean 31±10) and weighing from 57 and 85 kg (mean 71±14), participated in a standard 2 period, 2 sequence crossover study after providing written informed consent. All were judged to be healthy and were not receiving any medication during the study period. The volunteers were randomly divided into 2 groups of 6 each, and administered the preparations according to the schedule shown below. The orocaecal transit time was monitored by the co-administration of 250 mg of sulfasalazine to the volunteers at both phases.

Period

| Group | I | II |
|---|---|---|
| 1 | Artemisinin 250 | Artemisinin complexes |
| 2 | Artemisinin complexes | Artemisinin 250 |

On the first trial period, each volunteer in group 1 was given one capsule of Artemisinin 250 while those of group 2, one capsule of the artemisinin complexes. After a washout period of one week, each volunteer then received the alternate product. All products were administered in the morning (10.00 am) after an overnight fast. Food and drinks were withheld for at least 2 hours after dosing. Lunch and dinner comprising of chicken with rice, was served at 4 hours and 10 hours after dosing and water was given ad lib. All the volunteers were ambulatory during the trial and were prohibited from strenuous activity and consuming alcoholic beverages. Blood samples of 8 ml volume were collected in heparinized vacutainers (Becton Dickinson, New Jersey, USA) at 0 (before dosing), 20 min, /40 min, 1, 1.5, 2, 2.5, 3, 3.5, 4, 5, 6, 8, 10, 14 and 18 hours after dosing via an in-dwelling cannula placed in the forearm. The blood samples were centrifuged for 15 min at 2000 G and the plasma transferred to separate glass containers to be kept frozen until analysis. Sulfasalazine is hydrolyzed by the flora of the large intestine to produce sulfapyridine. Thus detection of the absorbed sulfapyridine in the blood would be an indication of the caecal arrival. Sulfapyridine levels in the plasma were analyzed using HPLC following the method of Yuen et al (1997). The protocol for the study was approved by a Joint School of Pharmaceutical Sciences, USM-General Hospital Penang Committee on Clinical studies. Volunteers were given information of the drug and the nature of the study in advance of the trial.

Plasma level of artemisinin was analyzed using a reversed-phase high performance liquid chromatography method employing electrochemical detection reported by Chan et al. (1997). The HPLC system comprised of a Jasco PU-980 solvent delivery system, a Digital Electrochemical Amperometric Detector (DECADE), a D-2500 Chromato-Integrator (Hitachi, Japan) and a Rheodyne 7725i injector fitted with a 20 μl sample loop. The column used was a Corsil CN-RP (5 μ 250×4.6 mm ID) (Bioscience, Malaysia) connected to refillable guard column (Upchurch Scientific, USA). The flow cell was equipped with a glassy carbon working electrode and Ag/AgCl reference electrode saturated with LiCl (BDH Chemicals Ltd, Poole, England). The mobile phase consisted of 75% of 0.01 M ammonium acetate (Merck, Darmstadt) buffer adjusted to pH 5.5 in acetonitrile (R & M Marketing, Essex, UK). Rigourous deoxygenation was performed by heating the mobile phase at 50° C. for 2 hours and then maintained at 30° C. while being purged with Argon. All the connections in the HPLC system are made of stainless steel. The system was left to stabilize for 24 hours prior to sample injection. The detector was operated in the reductive mode at an applied potential of −1.0 V and a sensitivity of 10 nA f.s. The signal was filtered at 0.1 s for the recorder output. Analysis was run at a flow rate of 1.4 ml/min and quantification was done by measuring the peak height ratio of the drug to the internal standard.

Prior to analysis, the plasma samples were treated using the following procedure: 1.0 ml of plasma was pipetted into a glass tube followed by the addition of 100 μl of 2 μg/ml dihydroartemisinin and 5 ml of tert-butylmethyl ether (Merck, Darmstadt). The mixture, was then vortexed for 1.5 minutes and centrifuged (Labofuge 200 Heraeus Sepatech GmbH, Germany) for 15 minutes at 3500 rpm. The supernatant was transferred into reactivial (Pierce Reacti-vial, USA) and evaporated with nitrogen gas at 35° C. The residue was reconstituted with 150 μl of mobile phase, vortexed and then transferred into an Eppendorf tube (Eppendorf, Germany). The mixture was centrifuged (Eppendorf Centrifuge 5410 Gmbh, Germany) at 12,800 g for 10 mins and the supernatant was transferred into 0.1 ml reaction vial (Pierce Reacti-vial, USA) for degassing for twenty minutes before being injected into the column.

The assay method was linear over a concentration range of 25.0 - 800.0 mg/ml and the detection limit was found to be 12.5 mg/ml at a signal to noise of 5:1. The accuracy was expressed as the percentage of the measured concentration over that of the spiked value whereas the precision was denoted using the coefficient of variation. Recovery values of the extraction procedure were calculated as a percentage of peak height obtained after extraction, over that of an equivalent amount of the drug without extraction. The within-day and between-day accuracy was found to be ±10.00% while the coefficient of variation was less than 7.00%. The mean recovery value of artemisinin was between 87.53% and 97.68% while the dihydroartemisinin (internal standard) had a mean recovery value of 94.93%.

The pharmacokinetic parameters, namely, maximum plasma concentration (Cₘₐₓ), time to reach maximum plasma concentration (Tₘₐₓ), and total area under the plasma concentration-time curve (AUC_{0-∞}), were estimated from the plasma concentration-time data. The values of Cₘₐₓ and Tₘₐₓ were obtained directly from the plasma values (Weiner, 1981). The AUC_{0-∞} was calculated by adding the area from time zero to time t (AUC₀₋ₜ) and the area from time t to infinity (AUC_{t-∞}). The former was calculated using the trapezoidal formula; and the latter by dividing the last measurable plasma drug concentration with the elimination rate constant (kₑ). In all cases, the AUC_{t-∞} was found to be less than 20% of the AUC_{0-∞}. The kₑ was estimated from the terminal slope of the individual plasma concentration-time curves after logarithmic transformation of the plasma concentration values and application of linear regression (Gibaldi and Perrier, 1982). The values of Cₘₐₓ, AUC_{0-∞} and kₑ obtained by the two preparations were analyzed using an analysis of variance (ANOVA) procedure which distinguishes effects due to subjects, periods, and treatment (Wagner, 1975). The AUC_{0∞} and Cₘₐₓ values were logarithmic transformed before analysis. On the other hand, the Tₘₐₓ values were analyzed using the Wilcoxon Signed Rank Test for paired samples.

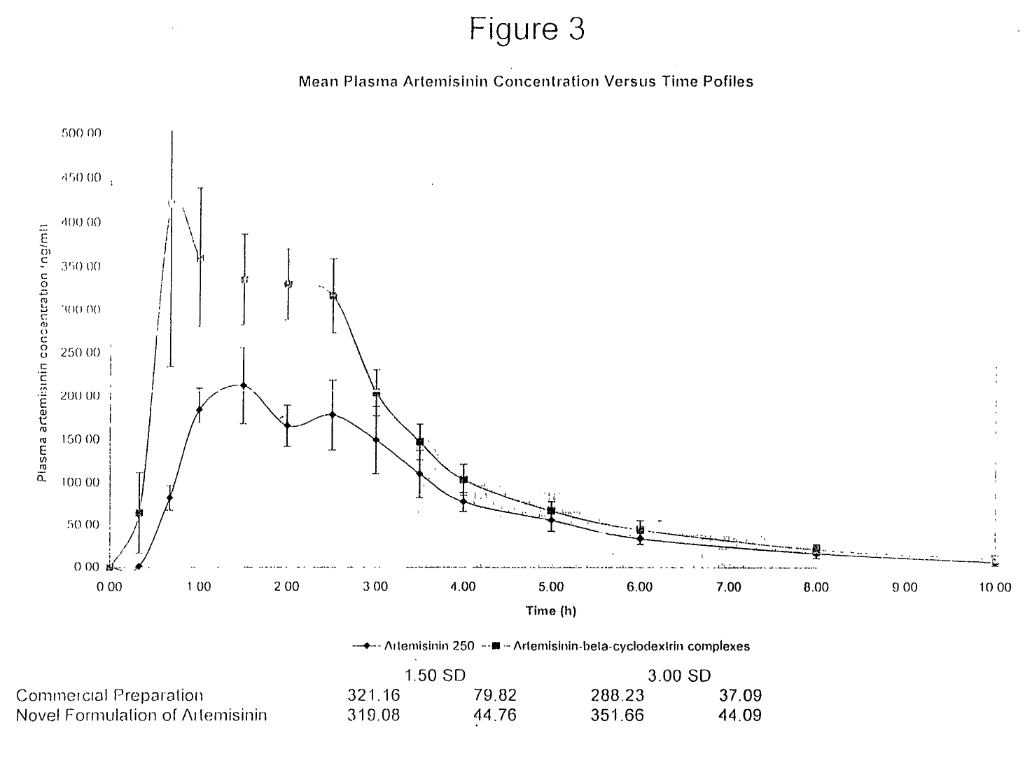

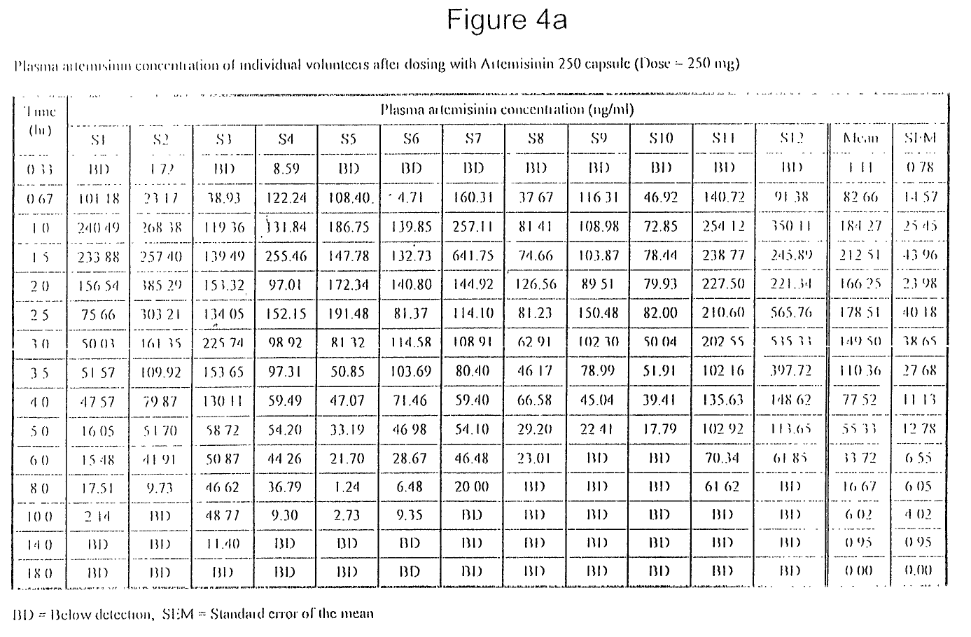

The mean plasma artemisinin concentration versus time profiles obtained with the complexes and Artemisinin 250 are as shown in FIG. 3, while the individual plasma concentration values are given in FIG. 4a and b. Referring to FIG. 3, a more rapid increase in plasma drug concentrations was observed with the complexes as compared to Artemisinin 250. The complexes achieved a peak plasma concentration at approximately 1.6 hours after dosing whereas Artemisinin 250 acheived a peak at about 2 hours, suggesting that the artemisinin-beta-cyclodextrin complexes had a faster rate of drug absorption. Moreover, the peak plasma concentration as well as the area under the plasma concentration versus time profile of the complexes was determined to be more than two times that of the Artemisinin 250, suggesting that the extent of absorption of artemisinin-beta-cyclodextrin complexes was two times higher.

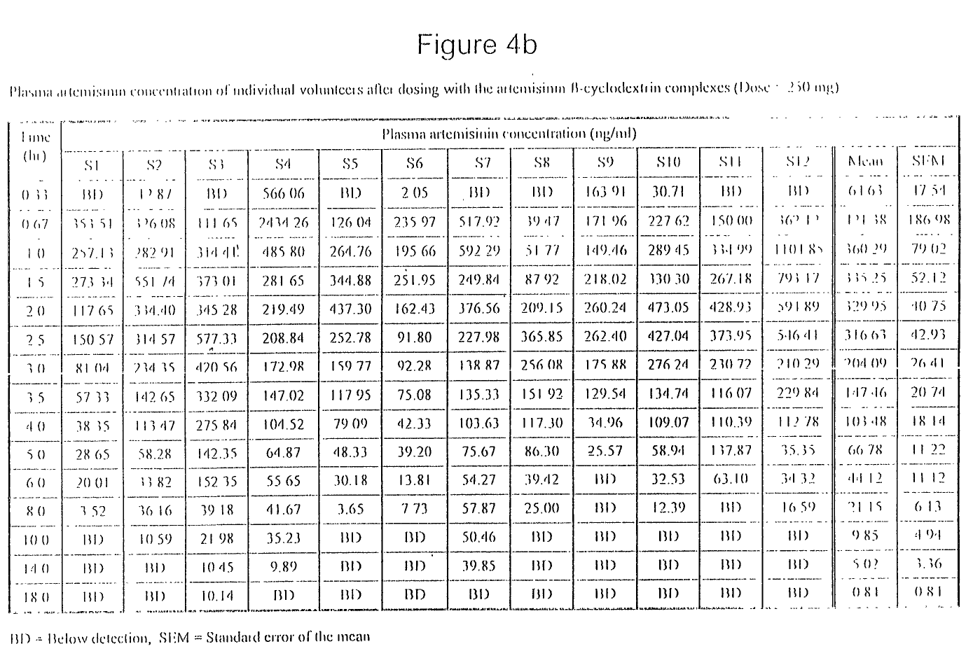

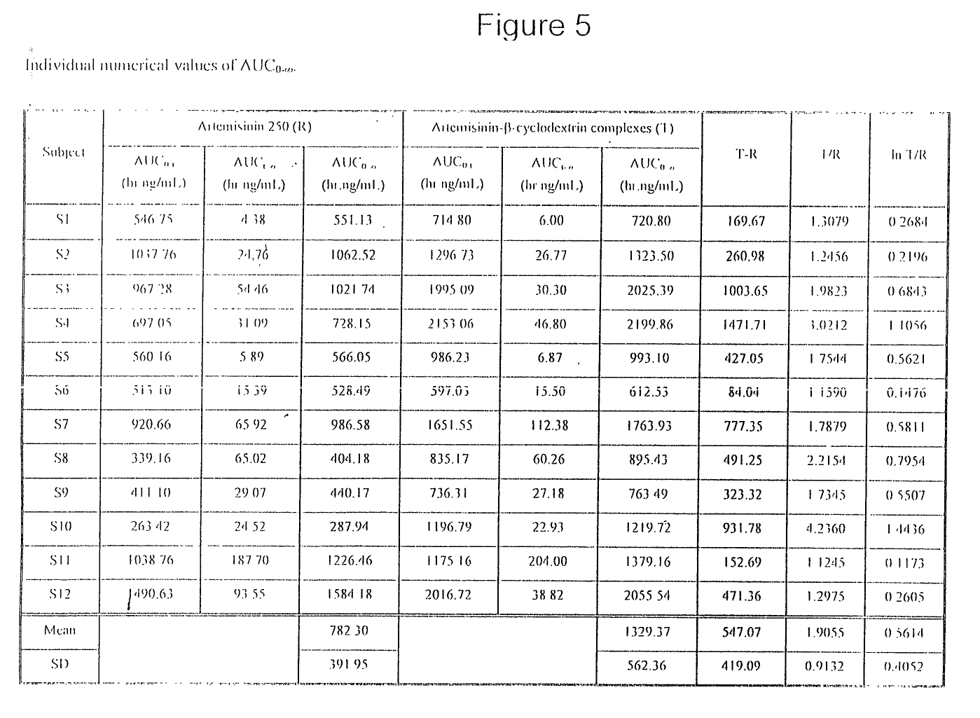

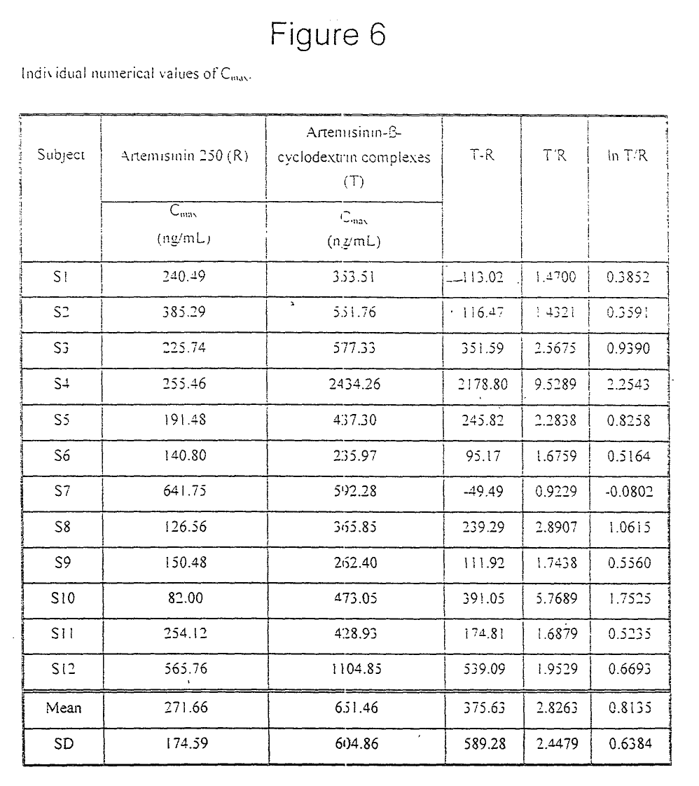

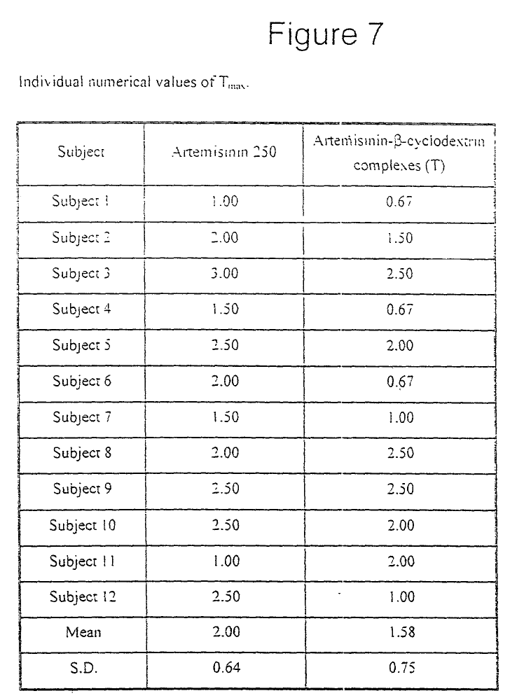

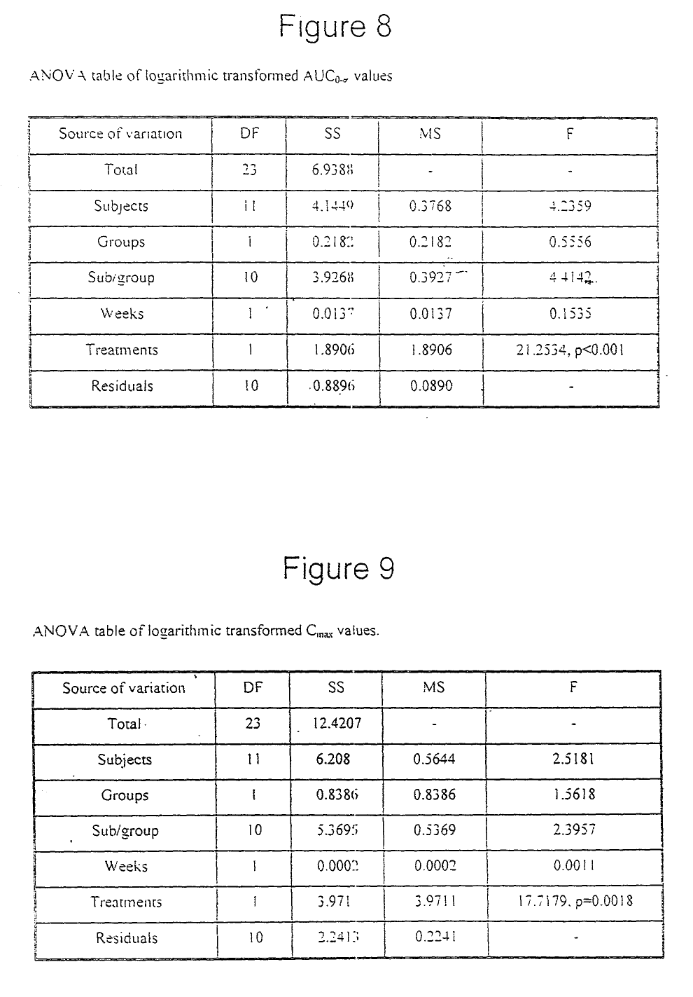

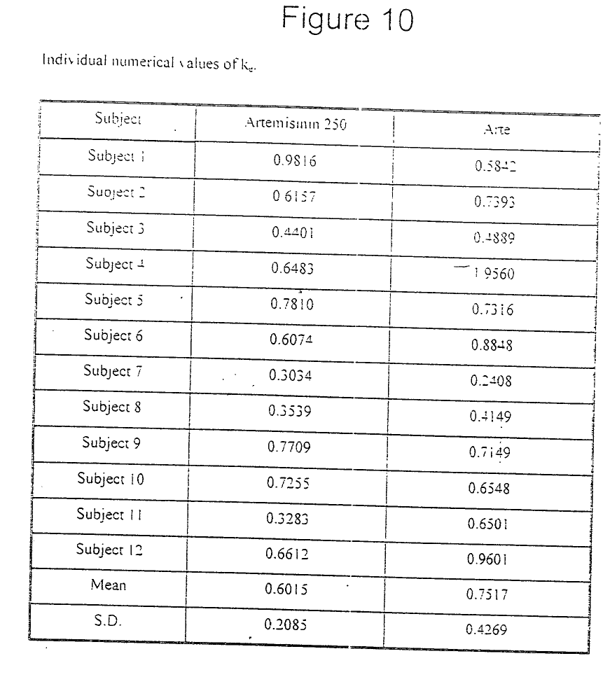

The numerical values of AUC_{0-∞} Cₘₐₓ and Tₘₐₓ obtained with the two preparations are as shown in FIG. 5, 6 and 7 respectively. The parameters Tₘₐₓ and AUC_{0-∞} are indicative of the respective rate and extent of drug absorption, whereas Cₘₐₓ is related to both processes (Grahnen, 1984). When the parameters were analyzed using the ANOVA procedure described previously, there was a statistically significant difference between the logarithmic transformed AUC_{0-∞} (p<0.001), as well as the logarithmic transformed Cₘₐₓ (p=0.0018) values of the two preparations. A summary of the statistical analyses is given in FIG. 8 and 9. The 90% confidence interval for the ratio of the logarithmic transformed AUC_{0-∞} values of the complexes over those of Artemisinin 250 was estimated to be between 1.4 -2.2, while that of Cₘₐₓ was between 1.61- 3.2. The results indicate that the complexes have a much better bioavailability in terms of both the rate and extent of absorption. Thus, artemisinin is absorbed faster and better from the complexes. In the case of the parameter Tₘₐₓ, there was also statistically significant difference (p<0.05) between the two preparations when analyzed using the Wilcoxon Signed Rank Test, in which the complexes needed a shorter time to reach peak plasma concentration. The kₑ values were also estimated from the individual plasma drug concentration profiles of the complexes and Artemisinin 250 and were given in FIG. 10. There was no statistically significant difference (p>0.05) between the values obtained with the two products. Moreover, the values obtained are comparable to those reported in the literature (Ashton et al. 1998, Titulaer et al. 1990).

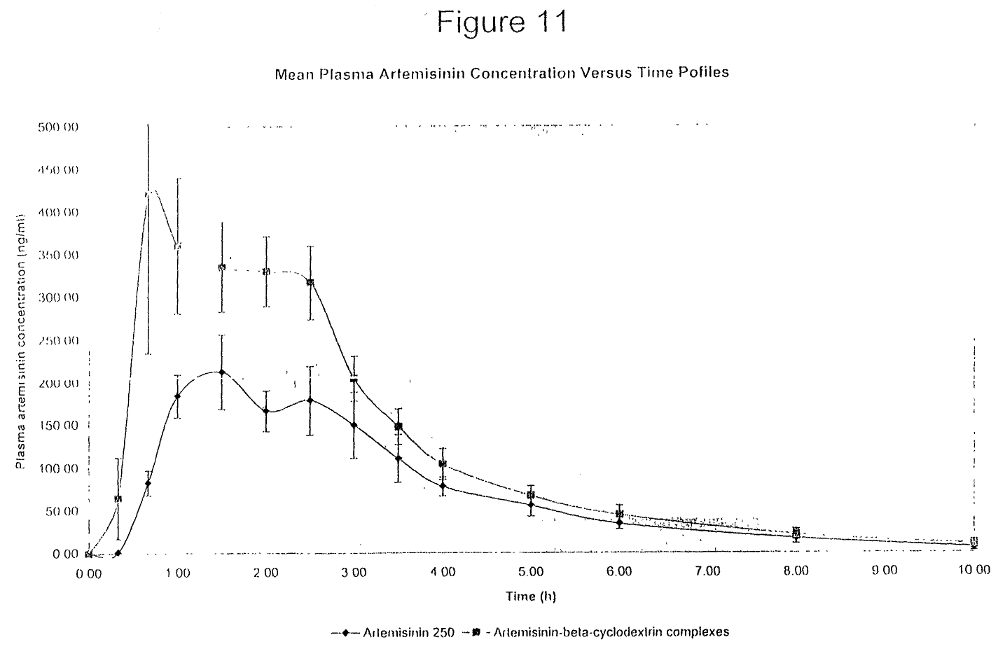

The absorption profiles of both preparations were also calculated using the Loo-Riegelman method (1968) which assumes a two-compartment model and were shown in FIG. 11. The absorption of artemisinin in the complexes was rapid and higher as compared to the commercial preparation. Moreover, there is a lag period in the absorption profile of the latter. However, both profiles showed that the absorption of artemisinin ceased at approximately 2.5 -2.7 hours.

The data obtained from measuring the plasma levels of sulfapyridine was used to estimate the orocaecal transit time (time to reach the colon) of both the formulations according to the method by Peh and Yuen (1996). A mean value of 2.7±0.6 hour was obtained with the commercial preparation while the patients in the complexes groups had a mean value of 2.5±0.7 hour. The value indicated the start of the arrival of the two preparations at the colon that coincided with the cessation of absorption of artemisinin. Thus, it appeared that the absorption of artemisinin was negligible in the colon. Therefore the lower extent of absorption observed with the commercial preparation was essentially due to less of its drug being dissolved for absorption prior to reaching the colon.

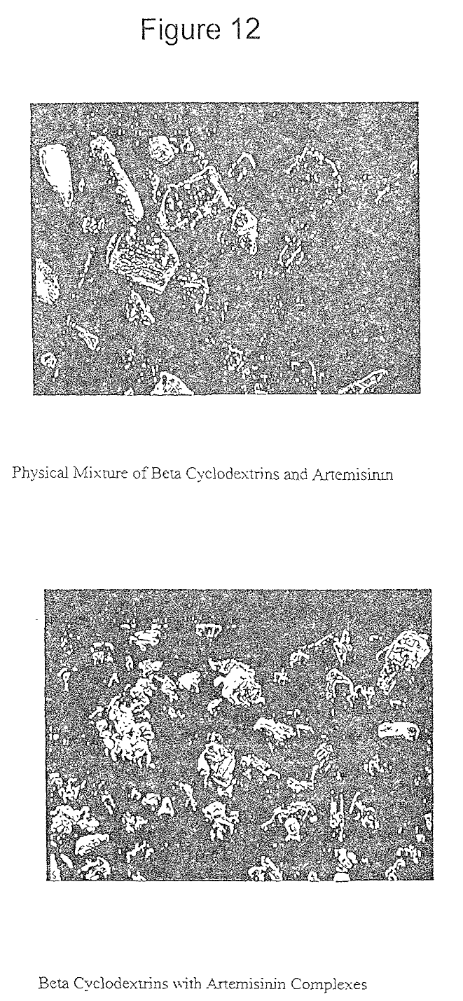

Microscopic examination of the complexes with the physical mixture revealed striking morphological differences. The micrographs of the complexes and physical mixtures are shown in FIG. 12. In the physical mixtures, the crystals of the individual beta cyclodextrins and artemisinin crystals were clearly visible, whereas in the formulation, there were very small particles which tend to agglomerate. This showed that the morphology of the components had changed during the process of producing the formulation. An artemisinin-beta-cyclodextrin complex is formed.

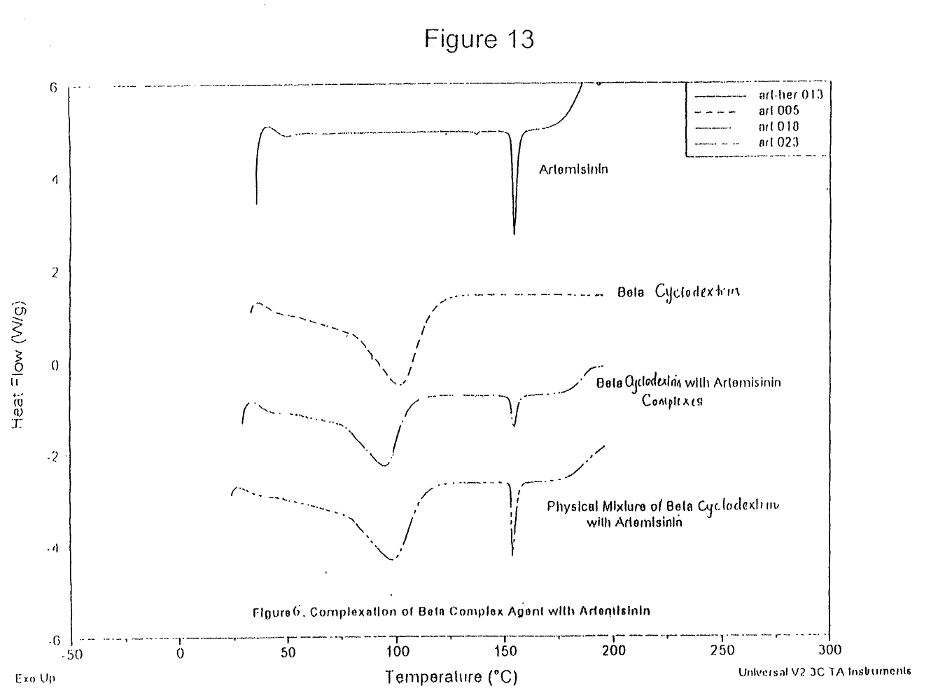

A TA differential scanning calorimeter was used to characterize the thermal behaviour of the complexes. The heating rate was set at 10° C./min over a range of 25° C. to 300° C. Samples of 2.5 mg each were placed in a hermetic aluminium pan with a pin-hole. Thermograms of the complexes and physical mixtures are shown in FIG. 13. Whilst pure artemisinin was characterized by the presence of a sharp melting peak with peak temperature at 152° C., the beta-cyclodextrins displayed a wide endothermic peak in the 80° C.-110° C. interval, which corresponded to the evaporation of water from the beta-cyclodextrins. The physical mixtures of artemisinin with beta-cyclodextrin were the superimposition of their own constituents, which indicated that no complexes were formed. On complexation, the melting endotherm of artemisinin showed a much smaller peak. This indicated that complexes had been formed. Moreover, the average percentages of complexed artemisinin had also been calculated, whereby 33.74% of artemisinin was encapsulated into the cavity of beta-cyclodextrins.

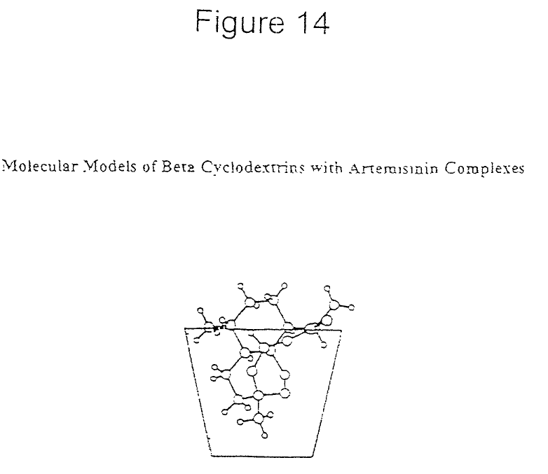

Molecular models of the beta-cyclodextrins with artemisinin complexes were obtained using the Corey-Pauling-Koltun model and are shown in FIG. 14. The artemisinin molecule was partially inserted into the beta-cyclodextrins and a tighter fitting complex is formed. Insertion was favoured towards the cycloheptane ring of the artemisinin molecule due to its narrower dimension as compared to the opposite end of the artemisinin molecule, consisting of two cyclohexane rings.

The complexes were further evaluated for their therapeutic efficacy using malarial patients. A study was conducted to determine the efficacy of a lower dose level of the novel formulation in comparison to a commercial preparation from Vietnam. Another aim was to determine if the novel formulation could circumvent the problem of recrudescence observed with the normal formulation (capsule) of the drug in view of its better and more consistent absorption. The study was carried out in Hospital Tawau, Semporna, Lahad Datu and Pusat Kesihatan Kunak, Apasbalung and Merotai. The study protocol was approved by the Ethics Committee and the Research Committee of the Ministry of Health Malaysia.

The study is conducted according to a single factor parallel group design with two treatment levels, that is:

Level 1:150 mg bd for 5 days with the novel formulation, given orally as capsule.

Level 2:250 mg bd for 5 days with commercial product, given orally as capsule.

58 patients were recruited for each treatment. This number was estimated based on the recommendation of Jones et al (1996) for an equivalence trial with α set at 0.05 and β at 0.2 (80% power).

The recommended dosage (250 mg bd×5 days) of the commercial preparation was reported to have 100% curing rate (WHO/MAL/94.1067, Malaria Unit, WHO, Geneva). Randomisation was done using a computer programme. All recruited patients were admitted into the hospital for seven days and paid a daily allowance of RM 10 per day (to be given on discharge). After discharge, they were monitored weekly for another 4 weeks. In this respect, the contact numbers and addresses of the patients were recorded and two laboratory technicians were assigned: to trace the patients after discharge.

Inclusion and exclusion criteria was based on WHO guidelines on the assessment of therapeutic efficacy of anti-malarial drugs (WHO/MAL/96.1077).

The inclusion criteria is listed below:

i. Ages between 15 and 60 years of both sexes.

ii. Absence of severe malnutrition.

iii. Mono-infection with Plasmodium falciparum, with a parasitaemia in the range of 2000 to 100,000 asexual parasites per μl.

iv. Absence of general danger signs or signs of severe and complicated falciparum malaria according to definition given by WHO.

V. Presence of axillary temperature >37.5° C. and <39.5° C. at admission.

vi. Absence of febrile conditions caused by diseases other than malaria.

vii. Ability to come for the stipulated follow-up visits, and easy access to the health facility.

viii. Informed consent of patient or parent/guardian of the patient.

The exclusion criteria is listed below:

i. Pregnant female patients.

ii. Patients with known renal and hepatic diseases.

iii. Patients on other anti-malarial therapy.

Three dependent variables were monitored:

a) Parasite clearance as determined using blood film were conducted twice daily for the first three days, daily for another 4 days followed by weekly monitoring for another 4 weeks. The clinical end point is total absence of parasites in blood film upon discharge on day 7. Otherwise treatment would be deemed unsuccessful. If the patient's blood film is cleared of parasite at the end of the 35th day, recrudescence is deemed to have not occurred. However, it should be pointed out that if parasites are detected on the fourth and fifth week, recrudescence could not be concluded since re-infection could not be excluded.

b) Fever subsidence. The body temperature of the patients was monitored at least 4 times (at predetermined time intervals) a day until day 7.

c) 5 ml blood samples were taken at 0 (predose), 1.5 and 3 hours after the administration of the first dose of the drug only. The plasma artemisinin concentration was determined using a HPLC method by Chan et al (1997).

The major parameter to monitor is the therapeutic outcome determined by the absence of parasite in the blood on day 7. The 95% confidence interval of the difference in the treatment outcomes of the two products will then be computed. Equivalence was to be accepted if the confidence interval lay within the set equivalence range of 90 to 100% (one sided test,-=10%). At the same time, the peak plasma drug concentration (estimated to be at approximately 1.5 hour from previous absorption studies) was to be compared between the two treatments to give an estimate of the rate and extent of drug absorption. The data was analyzed using a split-plot analysis of variance procedure (ANOVA) after logarithmic transformation. Similar analysis was also carried out on the 3-hour blood sample. The 90% confidence interval of the logarithmic transformed concentration value of the test preparation over that of the test product was computed. An estimation of the bioequivalence was made by comparison with the acceptable range of 0.80 to 1.25.

For a qualitative picture of the efficacy of the drug treatment, the parasite clearance and fever subsidence rate will be plotted and compared. Since the aim of the study was not to compare the rate of parasite clearance, no statistical analysis was carried out any further. Moreover, the initial parasite density may differ, thus a statistical comparison was not feasible.

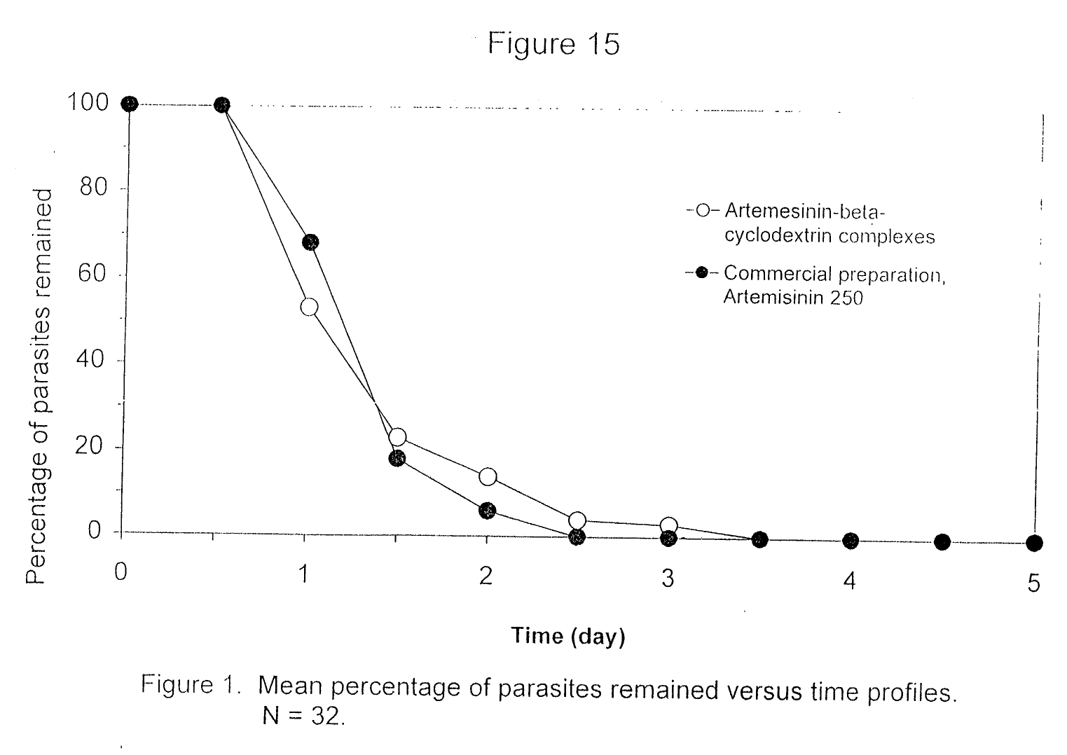

To date, 60 patients have participated in the study. All the sixty patients were found to be cleared of parasites rapidly and no parasites were detected at day 5. The percentage of parasites that remained in the blood versus time of the patients are show in FIG. 15. The curves indicating the parasite clearance rate for both preparations were almost superimposable. On the other hand, the mean fever subsidence versus time for the preparations are shown in FIG. 10. The mean duration for the body temperature of patients in both treatment arm to return to normal were approximately 2 days. In addition, the fever subsidence rate correlated well with the parasite clearance rate and the elevated body temperature returned to normal body temperature on day 5. The reduction of the body temperature was followed by a decrease of body temperature.

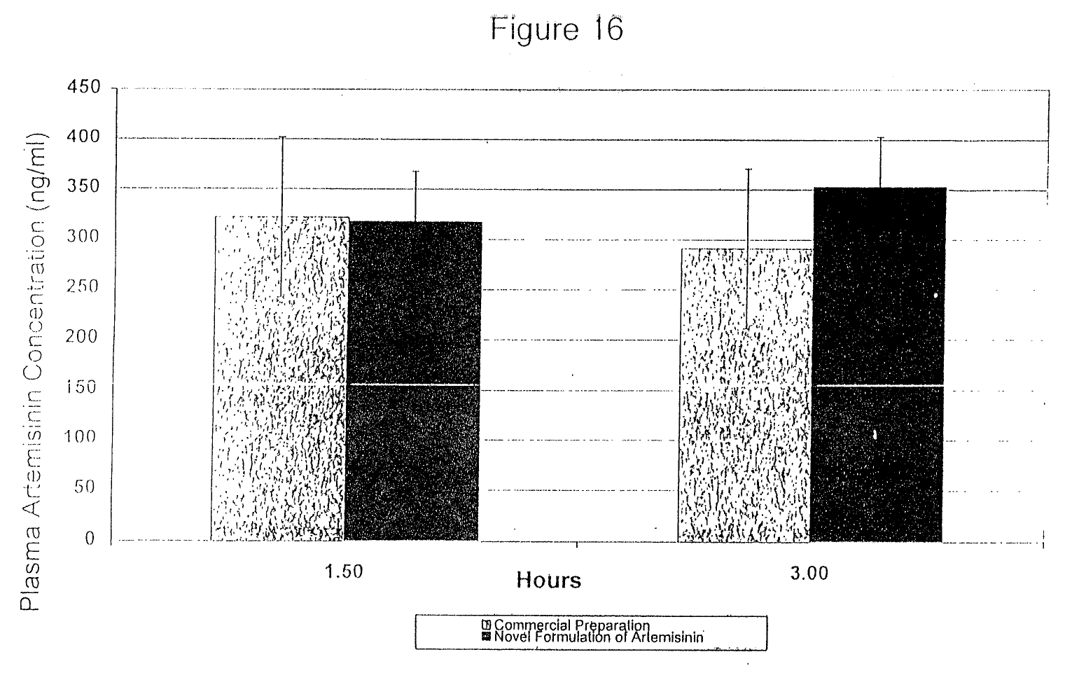

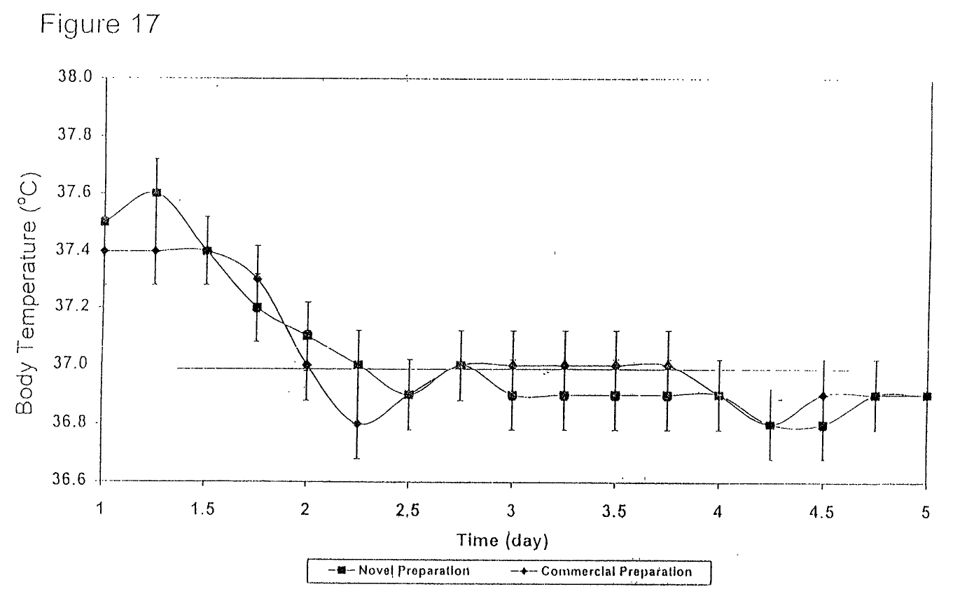

The mean concentration of the plasma level in patients receiving the complexes at 1.5 hour and 3 hour were 288.10 ng/ml (SEM=28.78) and 2,72.08 ng/ml (SEM=32.88), respectively whilst that of the patients receiving commercial preparation were 254.20 ng/ml (SEM=52.73) and 264.96 ng/ml (SEM=36.28), respectively. The plasma levels at both time intervals were comparable despite the fact that patients in the trial group received a lower dose (150 mg bd) as compared to the control group (250 mg bd). A histogram displaying the plasma levels were shown in FIG. 16. These results indicate that artemisinin-beta-cyclodextrins complexes (150 mg artemisinin) is equally effective in the treatment of malaria as compared to the commercial preparation (250 mg of artemisinin).

### REFERENCES

Ashton M, Gordi T, Trinh N H et al. (1998), Biopharm. Drug Disp., 19:245-250.

Chan K. L., Yuen K H and Sunil J et al. (1 997) Planta Med 63:66-69.

Gibaldi M and Perrier D (1982), Pharmacokinetics, 2nd ed., Marcel Dekker, New York, pp 145-195.

Grahnen A (1984), Pharmacy International, 5:100-103.

Higuchi T and Connors K A. (1965) Adv Anal Chem Instr 4 pp: 117-212.

Jones B, Jarvis P, Lewis J A and Ebbutt A F (1996), British Medical Journal 13:36-39.

Li Q G, Peggins J O and Fleckenstein L L et al. (1998) J Pharm Pharmcol 50:173-182.

Loo J C K and Riegelman S (1968), J Pharm Sci 57:918-928.

Olliaro P L and Trigg P I (1995) Bulletin of the World Health Organization 73(5):565-571.

Panisko D M and Keystone J S (1990) Drugs 39(2):160-189.

Peh K K and Yuen K H (1996), Int J Pharm 139:95-103.

Titulaer H A C, Zuidema J, Kager P A et al. (1990), J Pharm Pharmacol, 42: 810-813.

Wagner J G (1975), Fundamentals of Clinical Pharmacokinetics, 1st ed., Drug Intelligence, Hamilton, IL, pp 285-305.

Weiner W J (1981), In: Buncher C R and Tsay J Y (eds), Statistics in the Pharmaceutical Industry, Marcel Dekker Inc., New York, pp 205-229.

World Health Organization (1994) Report of an Informal Consultation. WHO/MAL/94. 1067.

World Health Organization (1996) WHO/MAL/96.1077.

Yuen K H, Peh K K and Quah Y L (1997) Drug Develop Indust Pharm 23(2):231-234.

1. A new formulation of artemisinin in the form of a complexation of artemisinin with beta-cyclodextrins.

2. A new formulation of artemisinin as claimed in claim 1 characterized as having greater aqueous solubility and higher dissolution rate.

3. A new formulation of artemisinin as claimed in claim 1 or 2 characterized in that it has improved bioavailability.

4. The use of the new formulation as claimed in claim 1 or 2 in the preparation of an oral pharmaceutical dosage form.

5. The pharmaceutical preparations at a dose of 150 mg as claimed in claim 4 which therapeutically equivalent to the commercial preparation at a dose of 250 mg.

6. The pharmaceutical preparations as claimed in claim 4 and 5 for use as an antimalarial drug.

7. A process for producing artemisinin in the form of a complexation of artemisinin with beta-cyclodextrins as claimed in claim 1 comprising of the following steps:
a) Mixing beta-cyclodextrins with distilled water.
b) Stirring the slurry of beta-cyclodextrins formed in step (a).
c) Adding finely ground artemisinin into the slurry.
d) Stirring the mixture formed in step (c) and then drying it at room temperature.
e) Grinding the dried product into fine powder and subsequently sieving it.

8. The process in accordance with claim 1 wherein:
a) The slurry in step (a) which consists of a ratio of 4 parts of beta-cyclodextrins to 5 parts of distilled water.
b) The slurry being stirred for 15 minutes.
c) In step (c) where 1 part of artemisinin (sieved through 300 μm mesh) is added into the slurry.
d) In step (d) where the mixture was stirred for 24 hours and dried by way of an extraction fan.
e) In step (e) the dried product being sieved through 300 μm mesh and the fine powder should have a loss on drying (LOD) of not more than 11.5%.
